(19) United States
(12) Patent Application Publication
(10) Pub. No.: US 2023/0054322 A1
(43) Pub. Date: Feb. 23, 2023

(54) Urine indication pad with inbuilt diagnostics for training and indication of potential disease

(71) Applicants: Leslie P. Taylor, Memphis, TN (US); Kristen M. Maurette, Memphis, TN (US)

(72) Inventors: Leslie P. Taylor, Memphis, TN (US); Kristen M. Maurette, Memphis, TN (US)

(21) Appl. No.: 17/409,016

(22) Filed: Aug. 23, 2021

(51) Int. Cl. A61B 5/00; A61F 13/42

(52) U.S. Cl. CPC A61B 5/6887; A61F 13/42; A61B 2503/40; A61B 2503/06; A61B 2560/0223; A61F 2013/422; A61F 2013/424; A61F 2013/425; A61F 2013/427

(57) ABSTRACT

A training pad system for house training of a pet or toilet training of a juvenile human, includes a training pad with one or more diagnostic zones, each comprising one or more diagnostic test areas, optionally a pad cutout that matches a shape of a lower portion of a toilet, a urine indicator composition for visually indicating exposure to urine with a signal color, a liquid absorbing material, an antimicrobial agent, and adhesive surfaces; and a diagnostic decoding chart to interpret diagnostic results by lookup of corresponding test decoding areas.

### CROSS-REFERENCE TO RELATED APPLICATIONS

N/A.

### FIELD OF THE INVENTION

The present invention relates generally to the field of training pads for pets and children, and more particularly to methods and systems for training pads with urine indication and diagnostic functions.

### BACKGROUND OF THE INVENTION

Current training pads for pets are made and used for the sole purpose of house breaking pets, and do not include any methods or devices for diagnosing disease conditions or even determine whether the pad has been used by the pet.

Additionally, while infections and illness are common in pets, such conditions may go undetected for extended periods of time, and most cases will remain undiagnosed until the pet is examined by a veterinarian.

As such, considering the foregoing, it may be appreciated that there continues to be a need for novel and improved devices and methods for the possible detection of illness or disease in domestic animals, elderly humans, and juvenile humans.

### SUMMARY OF THE INVENTION

The foregoing needs are met, to a great extent, by the present invention, wherein in aspects of this invention, enhancements are provided to the existing model of house pet training pads.

In an aspect, a training pad system for urine indication and diagnostics can include:
a) a training pad, including:
   i. a top layer, which can include:
      at least one diagnostic zone, which further can include:
         at least one diagnostic test area, which can be configured to indicate a diagnostic condition, after the at least one diagnostic test area is exposed to urine, such that a color change visually indicates a potential disease condition; and
      a urine indicator composition, for visual color indication of exposure to the urine;
   ii. a middle layer, which absorbs the urine; and
   iii. a bottom layer, which protects an underlying surface from exposure to the urine; and
b) a diagnostic decoding chart, which for each diagnostic test area in the at least one diagnostic test area can include a corresponding test decoding area, which can include:
   a negative test color calibration sample, which denotes a negative test result; and
   a positive test color calibration sample, which denotes a positive test result;
   such that diagnostic results shown by the diagnostic test areas of the diagnostic zone are interpreted by lookup of corresponding test decoding areas of the diagnostic decoding chart
such that the training pad can be used for house training of a pet, such as a dog, or a juvenile human.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. In addition, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1A is a perspective view of a training pad system, according to an embodiment of the invention.

FIG. 1B is a perspective view of a training pad system, according to an embodiment of the invention.

FIG. 2A is a top view of a training pad, according to an embodiment of the invention.

FIG. 2B is a top view of a training pad, according to an embodiment of the invention.

FIG. 2C is a bottom view of a training pad, according to an embodiment of the invention.

FIG. 3 is a cross sectional view of a training pad, according to an embodiment of the invention.

FIG. 4 is a schematic diagram illustrating a diagnostic decoding chart, according to an embodiment of the invention.

### DETAILED DESCRIPTION

Before describing the invention in detail, it should be observed that the present invention resides primarily in a novel and non-obvious combination of elements and process steps. So as not to obscure the disclosure with details that will readily be apparent to those skilled in the art, certain conventional elements and steps have been presented with lesser detail, while the drawings and specification describe in greater detail other elements and steps pertinent to understanding the invention.

The following embodiments are not intended to define limits as to the structure or method of the invention, but only to provide exemplary constructions. The embodiments are permissive rather than mandatory and illustrative rather than exhaustive.

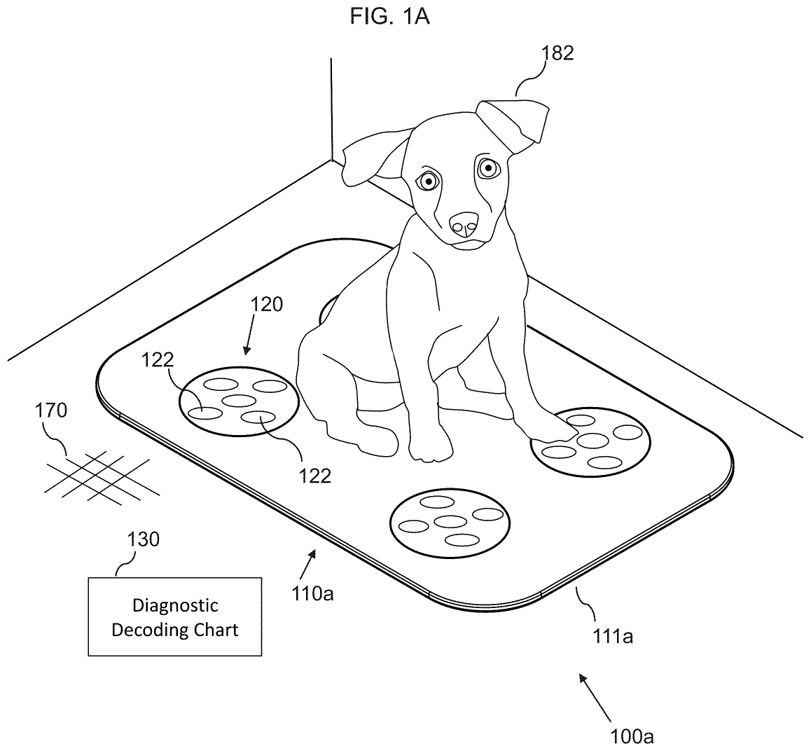

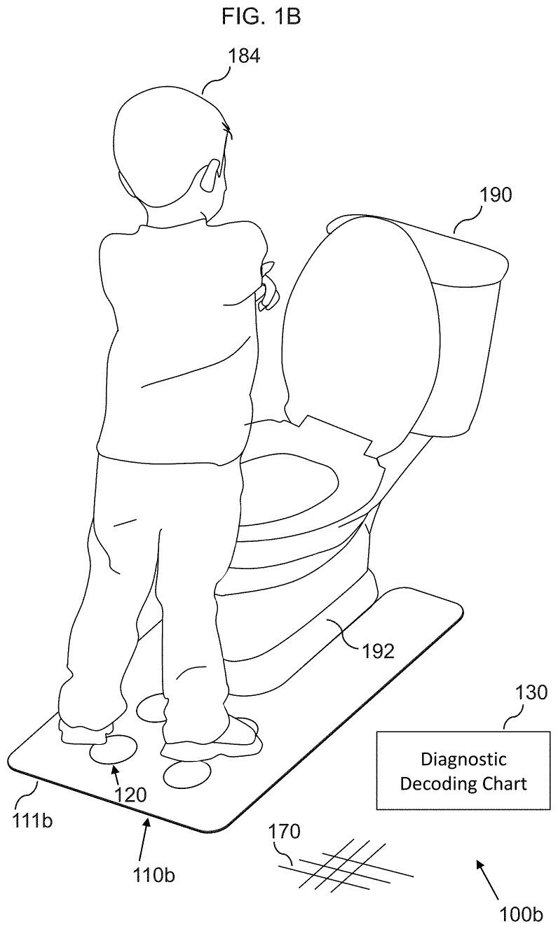

In the following, we describe the structure of an embodiment of a training pad 110a, 110b for absorbing and diagnosing illness in juvenile human and dog urine with reference to FIG. 1A, in such manner that like reference numerals refer to like components throughout; a convention that we shall employ for the remainder of this specification.

In an embodiment, as shown in FIGS. 1A and 1B, a training pad system 100a, 100b can include:
a) a training pad 110a, 110b, which is configured to receive (i.e., absorb) a liquid, such as urine, when the training pad is positioned on an underlying surface 170, such as a floor in a bathroom or other room of a house; or on a seat of a chair, such as a wheel chair, or car sear; or a on a mattress of a bed, or on another surface used by humans or pets;
   such that the training pad 110a, 110b can be used for house training of a pet 182, such as a dog 182, or a human 184, including a juvenile human, an elderly human, or a human patient with a medical condition.

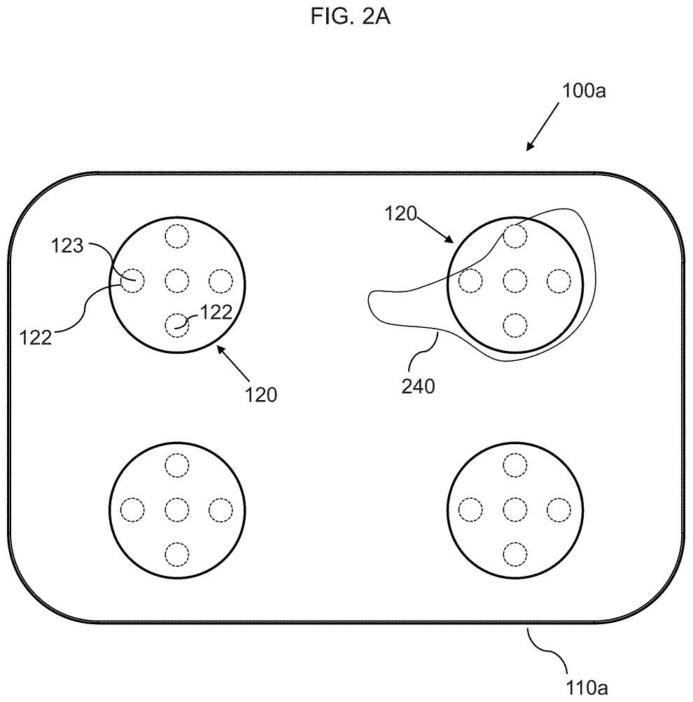

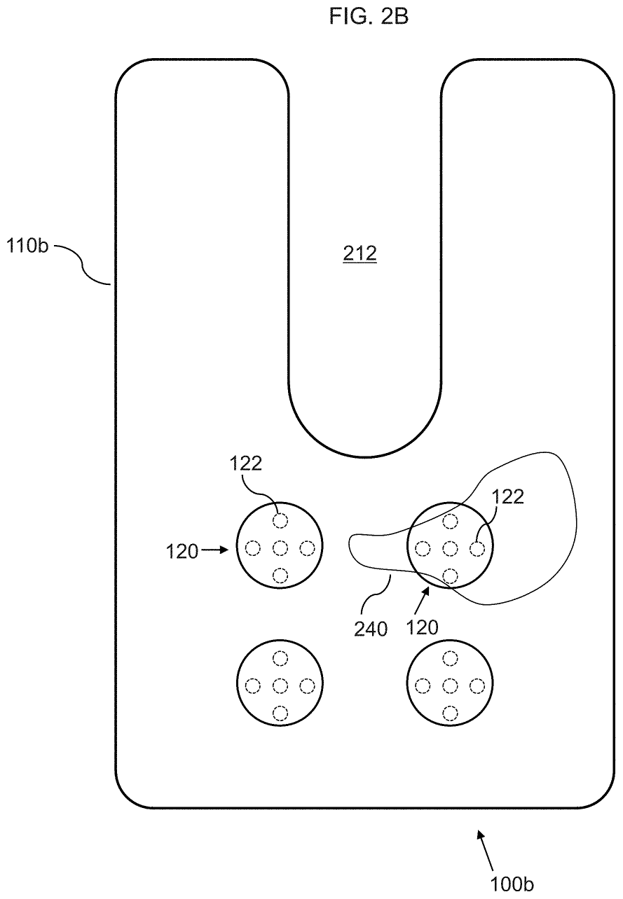

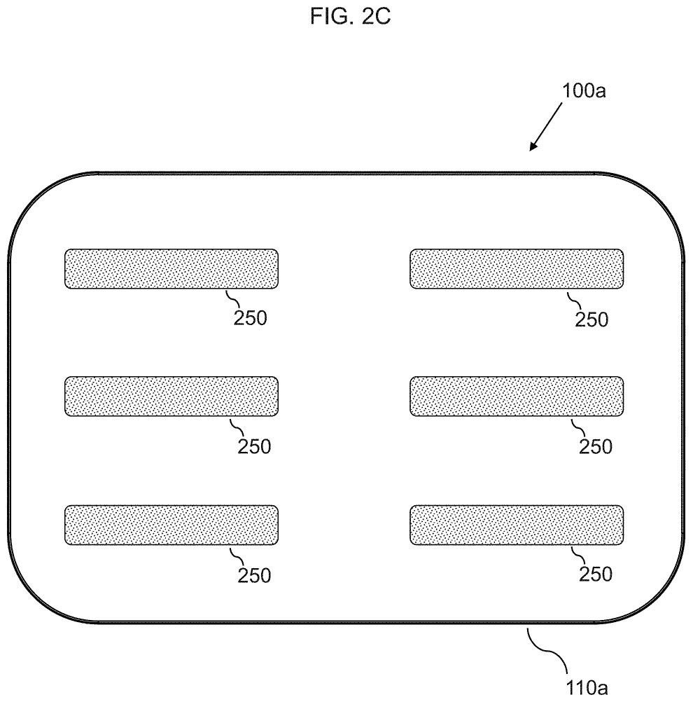

In a related embodiment, as shown in FIGS. 1A and 2A, the training pad 110a, 110b can further include at least one diagnostic zone 120, which can further include:
a) at least one diagnostic test area 122, which can be configured to indicate a diagnostic condition, after the at least one diagnostic test area 122 is exposed to urine 240 (i.e., saturated with urine), such that a color change visually indicates a potential disease condition;
wherein the at least one diagnostic zone 120 can be located on an upper surface 310 of the training pad 110a, 110b and is configured to visually indicate illness through coloration.

In a further related embodiment, the at least one diagnostic zone 120 can be indicated with a first printed line (or alternatively a first printed surface area) on a top surface of the training pad 110a, 110b, wherein the first printed line indicates an outer edge of the at least one diagnostic zone 120, and wherein the first printed line for example can be a colored line (or colored surface area), which indicates a location of the diagnostic test areas(s) 122.

In a yet further related embodiment, the at least one diagnostic test area 122 can be indicated with a second printed line (or alternatively a second printed surface area) on a top surface of the training pad 110a, 110b, within the at least one diagnostic zone 120, wherein the second printed line indicates an outer edge of the at least one diagnostic zone 120, and wherein the second printed line for example can be a colored line (or colored surface area), which indicates a location of the diagnostic test areas(s) 122.

In a related embodiment, as shown in FIG. 1A, the training pad 110a can be sized appropriately for domestic pet use, for example with a rounded rectangular shape as shown.

In a further related embodiment, the at least one diagnostic test area 122 can be configured (by predetermined calibration) with a calibrated concentration of the reactive agent 123 of the at least one diagnostic test area 122, such that the concentration of the reactive agent 123 is calibrated for exposure of the urine 240 until drying of the urine 240 has completed, such that exposure of urine 240 with a negative diagnostic condition will result in a color intensity up to or less than a negative threshold signal 446, and exposure of urine 240 with a positive diagnostic condition will result in a color intensity of at least or more than a positive threshold signal 448, wherein the positive threshold signal 448 is more color intense (i.e., has a stronger color) than the negative threshold signal 446. Note that conventional multi-panel test strips are typically calibrated for limited time exposure, such as 20 or 30 seconds, which generally permits more accurate reading and can distinguish an increased number of test value readings (rather than just negative or positive). The reduced accuracy of testing with increased length exposure (i.e. until completion of drying) means that the tests can normally be considered as only indicating a risk of disease, which warrants more accurate diagnostic testing to permit an actual diagnosis.

In another related embodiment, as shown in FIG. 1B, at least a portion of a training pad 110b can be configured to match a shape of a lower portion 192 of a toilet 190, such that the training pad covers a surface area surrounding the lower portion of the toilet, when the training pad 110b is positioned around the toilet 190, wherein the training pad 110b can further include:
a) a pad cutout 212, as shown in FIG. 2B, which is configured to (i.e. shaped to) match a shape of the lower portion 192 of a toilet 190, such that the training pad 110b covers a surface area surrounding front, left, and right sides of the lower portion 192 of the toilet 190, when the pad cutout 212 of the training pad 110b is positioned around the lower portion 192 of the toilet 190.

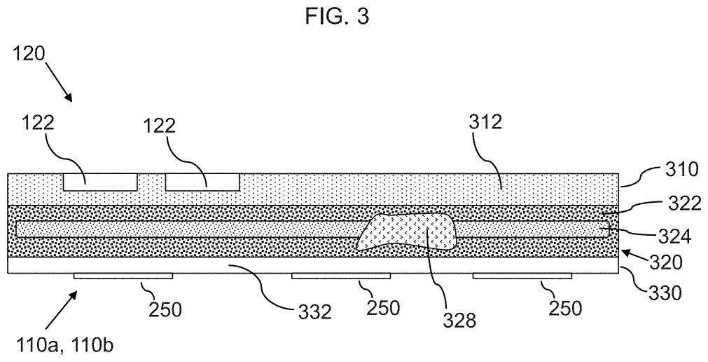

In an embodiment, as shown in FIG. 3, the training pad 110a, 110b can further include:
a) a top layer 310, which can comprise at least one or a plurality of diagnostic zones 120 and a urine indicator composition 312 throughout the surface of the top layer 310;
b) a middle layer 320, which is configured to absorb a liquid, such as urine, wherein the middle layer 320 can further include:
   i. a liquid absorbing material 322, such as a liquid absorbing gel or granulate; and
c) a bottom layer 330, which is waterproof, such that the bottom layer is configured to protect the surface from urine staining and discoloration;
wherein the top layer 310 can be positioned on top of the middle layer 320, which is positioned on top of the bottom layer 330, i.e. such that the middle layer 320 is vertically positioned between the top layer 310 and the bottom layer 330;
wherein the color changing element of the urine indicator composition 312 is on (or near) the surface of the training pad 110a, 110b.

In a related embodiment, all or at least portions of an upper surface of the top layer 310 of the training pad 110a, 110b, can further include:
a) a urine indicator composition 312, such that at least a portion of the upper surface comprises the urine indicator composition 312 (for example by impregnation of urine indicator composition 312), such that the urine indicator composition 312 is configured to change color from a base color to a signal color, when the urine indicator composition 312 is exposed to urine 240;
whereby the upper surface of the training pad 110a indicates a presence of urine by a color indication when the training pad is exposed to the urine, when a pet 182, such as a dog 182, or a juvenile human 184 has urinated on the training pad.

In a related embodiment, the urine indicator composition 312 can be a pH-indicating solution to provide a visually detectable color transition at a predetermined pH level, such that the urine indicator composition 312 changes from a base color to a signal color, such as red, when the urine indicator composition 312 is exposed to urine 240, such as described in U.S. Pat. No. 6,019,062, which is hereby incorporated herein by reference in its entirety.

In an embodiment, as shown in FIG. 3, the top layer 310 of the training pad 110a, 110b can indicate the presence of urine by the urine indicator composition 312 as well as the potential indication of infection or illness through the diagnostic zone 120, or the diagnostic test area 122.

In another related embodiment, the middle layer 320 of the training pad 110a, 110b can include an absorbent pad material 322, such as a foam, gel or granulate, to absorb the urine from the house pet 182 or juvenile human 184, after seeping through the top layer 310 and its diagnostic zone 120.

In a further related embodiment, the bottom layer 330 can be made from a material 332, such as a plastic sheet, to prevent any leakage (i.e., of urine) from escaping the bottom layer 330 and potentially staining or damaging the underlying floor surface 170.

In an embodiment, the training pad 110a, 110b can be configured to absorb urine 240, indicate possibility for infection or illness, and indicate exposure (or saturation) of urine by a color signal.

In an embodiment, the training pad 110a, 110b can be configured to identify if the domestic pet 182 or juvenile human 184 may potentially have an underlying disease or illness, indicate, and absorb urine at the time of initial contact (i.e., exposure) of urine with the training pad 110a, 110b.

In an embodiment, the training pad 110a, 110b can include urine reagent tests on the top layer 310, which can include at least one or a plurality of diagnostic test areas 122, such as 1-14 diagnostic test areas 122, which are configured to test respectively 1 to 14 parameters. The diagnostic test areas 122 can for example be configured to test, individually or jointly, at least one or a combination of: Ketone, pH, Leukocytes, Urobilinogen, Microalbumin, Protein, Bilirubin, Glucose, Ascorbic Acid, Specific Gravity, Nitrite, Creatinine, Blood, and Calcium, in urine deposited on the chemical pads. Such diagnostics are well-known and commercially available reagent diagnostic tests 122 may have a variety of product specifications, with variant color transitions and required reaction times.

In a further related embodiment, the top layer 310 of the training pad 110a, 110b can include urine reagent tests, comprising at least one diagnostic test area 122, which is selected from the group consisting of:
a) a ketone test diagnostic test area 122;
b) a pH test diagnostic test area 122;
c) a leukocytes test diagnostic test area 122;
d) a urobilinogen test diagnostic test area 122;
e) a microalbumin test diagnostic test area 122;
f) a protein test diagnostic test area 122;
g) a bilirubin test diagnostic test area 122;
h) a glucose test diagnostic test area 122;
i) an ascorbic acid test diagnostic test area 122;
j) a specific gravity test diagnostic test area 122;
k) a nitrite test diagnostic test area 122;
l) a creatinine test diagnostic test area 122;
m) a blood test diagnostic test area 122, for testing for a presence of blood in the urine; and
n) calcium test diagnostic test area 122.

In an embodiment, a training pad 110b, which is configured with a pad cutout 212, as shown in FIG. 2B, which is configured to (i.e. shaped to) match a shape of the lower portion 192 of a toilet 190, can be configured without any diagnostic test area 122, and can optionally include a urine indicator composition 312, such that the training pad 110b can be used for toilet training for juvenile humans.

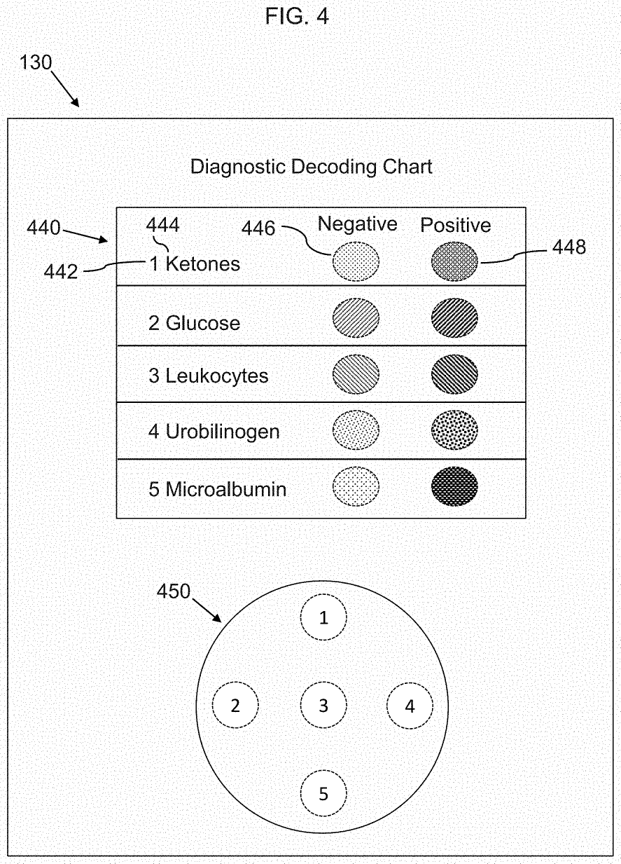

In an embodiment, as shown in FIG. 4, a diagnostic decoding chart 130 can be configured to interpret what the diagnostic test areas 122 (as shown in FIGS. 2A and 2B) indicate after exposure to urine; for example, to indicate if the diagnostic test area 122 results are in a healthy range, or if the results may be indicating a possibility of illness or infection.

In a related embodiment, as shown in FIG. 4, the training pad system 100a, 100b can further include:
a) a diagnostic decoding chart 130, which can be a physical printed chart or a digital chart for viewing on a computer device, such as a PC, tablet, or smartphone, wherein the diagnostic decoding chart 130 for each diagnostic test area 122 in the at least one diagnostic zone 120 can include a corresponding test decoding area 440, which can include:
   i. a test number 442;
   ii. a test description 444;
   iii. a negative test color calibration sample 446, which denotes a negative test result; and
   iv. a positive test color calibration sample 448, which denotes a positive test result;
      such that diagnostic results (as shown by the diagnostic test areas 122) of a diagnostic zone 120 can be interpreted by lookup of corresponding test decoding areas 440 of the diagnostic decoding chart 130.

In a further related embodiment, the diagnostic decoding chart 130 can further include a symbolic legend 450, which graphically, symbolically and/or textually describes locations of each numbered 442 diagnostic test area 122 within a diagnostic zone 120. The symbolic legend 450 can be separate, as shown in FIG. 4, or alternatively be an integrated part of the test decoding areas 440.

In a related embodiment, as shown in FIGS. 2C and 3, a bottom surface of the training pad 110a, 110b can further include:
a) at least one adhesive surface 250, such as a plurality of adhesive strips 250, which for example each can be configured as a pressure sensitive adhesive gel or a double sided adhesive tape;
wherein the at least one adhesive surface 250 can be configured to allow removable attachment of the training pad 110a, 110b to the underlying surface 170.

In a related embodiment, as shown in FIG. 3, the middle layer 320 (and/or other parts) of the training pad 110a, 110b can further include:
a) an antimicrobial agent 324;
which is configured to inhibit microbial growth 328 in the training pad 110a, 110b, such as growth of fungi and bacteria, which can otherwise be stimulated by the presence of urine 240. The antimicrobial agent 324 can be intermixed/combined with the liquid absorbing material 322.

In a further related embodiment, the antimicrobial agent 324 can include a quaternary ammonium salt, silver particles, copper particles, other well-known antimicrobial agents, and compositions thereof; and can for example be formulated as a powder or granulate.

In various related embodiments, the training pad 110a, 110b can alternatively be described such that the training pad 110a, 110b can include:
a) a training pad body 111a, 111b, which can be configured with a pad cutout 212, wherein the training pad body 111a, 111b can include:
   i. a top layer 310;
   ii. a middle layer 320; and
   iii. a bottom layer 330; and
b) at least one diagnostic zone 120, positioned in the training pad body 111a, 111b, wherein the at least one diagnostic zone 120 can include:
   i. at least one diagnostic test area 122.

Here has thus been described a multitude of embodiments of the training pad system 100a, 100b, and methods related thereto, which can be employed in numerous modes of usage, including for absorbing urine, indicating urine, and diagnosing potential disease.

The many features and advantages of the invention are apparent from the detailed specification, and thus, it is intended by the appended claims to cover all such features and advantages of the invention, which fall within the true spirit and scope of the invention.

Many such alternative configurations are readily apparent and should be considered fully included in this specification and the claims appended hereto. Accordingly, since numerous modifications and variations will readily occur to those skilled in the art, the invention is not limited to the exact construction and operation illustrated and described, and thus, all suitable modifications and equivalents may be resorted to, falling within the scope of the invention.

What is claimed is:

1. A training pad system, comprising:
a training pad, which is configured to receive urine when the training pad is positioned on a surface, wherein the training pad further comprises:
at least one diagnostic test area, which is configured to indicate a diagnostic condition, after the at least one diagnostic test area is exposed to the urine, such that a color change visually indicates a potential disease condition.

2. The training pad system of claim 1, wherein the training pad further comprises:
at least one diagnostic zone, which further comprises:
a plurality of diagnostic test areas.

3. The training pad system of claim 1, wherein a portion of the training pad is configured to match a shape of a lower portion of a toilet, such that the training pad covers a surface area surrounding the lower portion of the toilet, when the training pad is positioned around the toilet.

4. The training pad system of claim 3, wherein the training pad further comprises:
a pad cutout, which is configured to match the shape of the lower portion of the toilet, such that the training pad covers the surface area surrounding front, left, and right sides of the lower portion of the toilet, when the training pad is positioned around the toilet.

5. The training pad system of claim 1, wherein the training pad further comprises:
a) a top layer, which comprises the at least one diagnostic test area;
b) a middle layer, which is configured to absorb the urine; and
c) a bottom layer, which is waterproof, such that the bottom layer is configured to protect the surface from the urine;
wherein the middle layer is vertically positioned between the top layer and the bottom layer.

6. The training pad system of claim 5, wherein the middle layer further comprises:
a liquid absorbing material.

7. The training pad system of claim 5, wherein the middle layer further comprises:
an antimicrobial agent, which is configured to inhibit microbial growth in the training pad.

8. The training pad system of claim 7, wherein the antimicrobial agent comprises a quaternary ammonium salt.

9. The training pad system of claim 1, wherein an upper surface of the training pad further comprises:
a urine indicator composition, such that at least a portion of the upper surface comprises the urine indicator composition, such that the urine indicator composition is configured to change color to a signal color when the urine indicator composition is exposed to the urine;
whereby the upper surface of the training pad indicates a presence of the urine by the signal color when the training pad is exposed to the urine.

10. The training pad system of claim 9, wherein the urine indicator composition is a pH-indicating solution which is configured to provide a visually detectable color transition at a predetermined pH level.

11. The training pad system of claim 2, further comprising:
a diagnostic decoding chart, which for each diagnostic test area in the at least one diagnostic zone comprises a corresponding test decoding area, which comprises:
a negative test color calibration sample, which denotes a negative test result; and
a positive test color calibration sample, which denotes a positive test result;
such that diagnostic results shown by the diagnostic test areas of the diagnostic zone are interpreted by lookup of corresponding test decoding areas of the diagnostic decoding chart.

12. The training pad system of claim 1, wherein the at least one diagnostic test area is configured with a calibrated concentration of a reactive agent of the at least one diagnostic test area, such that the calibrated concentration of the reactive agent is calibrated for exposure of the urine until drying of the urine has completed, such that exposure of the urine with a negative diagnostic condition will result in a color intensity up to a negative threshold signal, and exposure of the urine with a positive diagnostic condition will result in the color intensity of at least a positive threshold signal.

13. The training pad system of claim 1, wherein a bottom surface of the training pad further comprises:
at least one adhesive surface;
wherein the at least one adhesive surface is configured to allow removable attachment of the training pad to the surface.

14. A training pad system, comprising:
a training pad, which is configured to receive urine when the training pad is positioned on a surface;
such that the training pad is configured to be used for house training of a pet or toilet training of a juvenile human.

15. The training pad system of claim 14, wherein the training pad further comprises:
at least one diagnostic test area, which is configured to indicate a diagnostic condition, after the at least one diagnostic test area is exposed to the urine, such that a color change visually indicates a potential disease condition.

16. The training pad system of claim 14, wherein the training pad further comprises:
at least one diagnostic zone, which further comprises:
at least one diagnostic test area, which is configured to indicate a diagnostic condition, after the at least one diagnostic test area is exposed to the urine, such that a color change visually indicates a potential disease condition.

17. The training pad system of claim 14, wherein a portion of the training pad is configured to match a shape of a lower portion of a toilet, such that the training pad covers a surface area surrounding the lower portion of the toilet, when the training pad is positioned around the toilet.

18. The training pad system of claim 17, wherein the training pad further comprises:
a pad cutout, which is configured to match the shape of the lower portion of the toilet, such that the training pad covers the surface area surrounding front, left, and right sides of the lower portion of the toilet, when the training pad is positioned around the toilet.

19. The training pad system of claim 15, wherein the training pad further comprises:
a) a top layer, which comprises the at least one diagnostic test area;
b) a middle layer, which is configured to absorb the urine; and
c) a bottom layer, which is waterproof, such that the bottom layer is configured to protect the surface from the urine;
wherein the middle layer is vertically positioned between the top layer and the bottom layer.

20. The training pad system of claim 19, wherein the middle layer further comprises:
a liquid absorbing material.

21. The training pad system of claim 14, wherein an upper surface of the training pad further comprises:
a urine indicator composition, such that at least a portion of the upper surface comprises the urine indicator composition, such that the urine indicator composition is configured to change color to a signal color when the urine indicator composition is exposed to urine;
whereby the upper surface of the training pad indicates a presence of the urine by the signal color when the training pad is exposed to the urine.

22. The training pad system of claim 21, wherein the urine indicator composition is a pH-indicating solution which is configured to provide a visually detectable color transition at a predetermined pH level.

23. The training pad system of claim 16, further comprising:
a diagnostic decoding chart, which for each diagnostic test area in the at least one diagnostic zone comprises a corresponding test decoding area, which comprises:
a negative test color calibration sample, which denotes a negative test result; and
a positive test color calibration sample, which denotes a positive test result;
such that diagnostic results shown by the diagnostic test areas of the diagnostic zone are interpreted by lookup of corresponding test decoding areas of the diagnostic decoding chart.

24. The training pad system of claim 15, wherein the at least one diagnostic test area is configured with a calibrated concentration of a reactive agent of the at least one diagnostic test area, such that the calibrated concentration of the reactive agent is calibrated for exposure of the urine until drying of the urine has completed, such that exposure of the urine with a negative diagnostic condition will result in a color intensity up to a negative threshold signal, and exposure of the urine with a positive diagnostic condition will result in the color intensity of at least a positive threshold signal.

25. A training pad, which is configured to receive urine when the training pad is positioned on a surface, wherein an upper surface of the training pad further comprises:
a urine indicator composition, such that at least a portion of the upper surface comprises the urine indicator composition, such that the urine indicator composition is configured to change color to a signal color when the urine indicator composition is exposed to urine;
whereby the upper surface of the training pad indicates a presence of the urine by the signal color when the training pad is exposed to the urine;
such that the training pad is configured to be used for house training of a pet or toilet training of a juvenile human.

26. The training pad of claim 25, wherein a portion of the training pad is configured to match a shape of a lower portion of a toilet, such that the training pad covers a surface area surrounding the lower portion of the toilet.

27. The training pad of claim 26, wherein the urine indicator composition is a pH-indicating solution which is configured to provide a visually detectable color transition at a predetermined pH level.
